(19) United States
(12) Patent Application Publication
(10) Pub. No.: US 2005/0095230 A1
(43) Pub. Date: May 5, 2005

(54) Interleukin-2 gene transferred lymphokine activated killer cells

(76) Inventors: Nae Choon Yoo, Seoul (KR); Kyung Hee Chang, Seoul (KR); Yeon Soo Kim, Daejeon (KR); June Myung Kim, Seoul (KR)

Correspondence Address: JHK Law, PO Box 1078, La Canada, CA, 91012-1078, US

(21) Appl. No.: 10/495,900

(22) PCT Filed: Nov. 22, 2002
(86) PCT No.: PCT/KR02/02196

(30) Foreign Application Priority Data
Nov. 22, 2001 (KR) 2001-0073136

(51) Int. Cl. A61K 48/00; C12N 5/08; C12N 15/861

(52) U.S. Cl. 424/93.21; 435/372; 435/456; 514/44

(57) ABSTRACT

The present invention relates to a transformed lymphokine activated killer cell (I-LAK cell) useful in the treatment of cancer or virus infection into which a recombinant vector carrying IL-2 gene to which it is operably linked is introduced. Also it relates to a pharmaceutical composition for treating cancer or virus infection disease comprising a sufficient amount of I-LAK cell to elicit an immune response.

The present application is a U.S. national phase application under 35 U.S.C. §371, of PCT/KR02/02196, filed Nov. 22, 2002.

### TECHNICAL FIELD

The present invention relates to a transformed lymphokine activated killer cell for the treatment of cancer or virus infection into which interleukin-2 gene is introduced. More particularly, the present invention relates to a transfected lymphokine activated killer cell with a recombination vector carrying interleukin-2 gene, a process for producing the transfected cell by transfecting a lymphokine activated killer cell derived from mammalian peripheral blood with a recombinant vector carrying interleukin-2 gene, and a pharmaceutical composition for the treatment of cancer or virus infection comprising the transfected cell. The transfected lymphokine activated killer cell of the present invention has the characteristic property that it constantly secrets low concentration of interleukin-2 and exhibits activity to selectively destroy cancer cells and virus-infected cells.

### BACKGROUND ART

A lymphokine activated killer cell (hereinafter referred to as LAK cell) is generally used in adoptive immunotherapy for the treatment of malignant melanoma and colon cancer, etc. The adoptive immunotherapy using LAK cells is for the purpose of the treatment especially of cancer in which lymphocytes are removed from a patient, cultured with interleukin-2 (IL-2) to induce their transformation into LAK cells, and returned to the patient's body along with interleukin-2.

The adoptive immunotherapy has been advanced during the latest 10 years and is focusing to directions that restores innate immune functions or enhances resistance of patients to cancer. Generally, it has been known that the adoptive immunotherapy is effective for the treatment of cancer relatively strongly resistant to immunity, for example, malignant melanoma, etc.

Immunotherapeutic agents commonly used in the immunotherapy other than LAK cells include BCG, interferon, interleukin-2, tumor necrosis factor, monoclonal antibody, Picibanil, Helix, etc. BCG, an attenuated strain of tuberculosis, was used at the initial time of immunotherapy and it has been known that BCG is effective against malignant melanoma by reinforcing the function of phagocyte to digest cancer cells and enhancing lymphocytes cell-mediated immunity. Interferon causes a variety of adverse side effects by affecting normal cells in addition to cancer cells and thereby is hardly used at present.

A process for producing LAK cells is disclosed for example in U.S. Pat. No. 4,849,329 to Leung.

U.S. Pat. No. 5,108,760 to Irr discloses a process for enhanced LAK cell activation wherein the peripheral blood mononuclear cells are treated with an amino acid amide and a pharmaceutical composition for the treatment of cancer containing said LAK cells.

U.S. Pat. No. 4,690,915 to Rosenberg discloses a method for treating cancer in humans by co-administrating LAK cells and IL-2.

Meanwhile, LAK cells can be also used in the treatment of HIV infection and this study has been performed in vitro. There have been provided evidences that LAK cells are effective for the in vitro removal of HIV-infected cells (Tyler D S et al., J. Surg. Res., 79, 115-120 (1998); and Wang L., et al., Virology, 241, 169-180 (1998)).

Although the treatment of HIV infection has been developed rapidly for recent years, HIV infection is not yet cured finely up to now. Among therapies developed until now, co-administration of at least 3 antiretroviral agents, called HAART (highly active antiretroviral therapy), represents a standard therapy of HIV infection (Hammer S. et al., N. Engl. J. Med., 337, 725-733 (1997); and Staszewski S., N. Engl. J. Med., 341, 1865-1873 (1999)). However, HAART is limit to cure HIV infection completely in that antiretroviral agents neither work on latent HIV cells nor pass anatomical barriers such as cerebral vascular and testicular vascular barriers. Alternative HIV therapy is co-administration of IL-2 in combination of the above antiretroviral agents (Chun T W et al., Nature Med., 5(6), 651-655 (1999)). Likewise, this therapy is also not sufficient to cure HIV infection.

In the treatment of cancer and HIV infection using LAK cells, several problems should be overcome in order to apply LAK cells clinically. LAK cells would be routinely injected along with high concentration of IL-2 to keep alive in the body. It has been reported hat high concentration of IL-2 instantly activates HIV virus in blood (viral blips) and secondly increases latent cells (Ramratnam B et al., Nat Med, 6(1), 82-5 (2000)). In addition, there has been suggested another problem that continuous administration of high concentration of IL-2 results in a variety of adverse side effects including cytokine leak syndrome.

We prepared a transformed lymphokine activated killer cell with a recombinant vector carrying IL-2 gene and found that the transformed cell constantly secretes low concentration of IL-2 and selectively destroys cancer cells or virus-infected cells while hardly affecting normal cells.

### BRIEF DESCRIPTION OF THE DRAWINGS

FIG. 1 shows a schematic diagram of the recombinant vector LNC/IL-2/IRES/TK according to the present invention. (LNC: LTR promoter/neomycin marker/CMV promoter; IRES: internal ribosome entry site, TK: thymidine kinase; MSV: mouse sarcoma virus; MLV: mouse leukaemia virus; SD: splicing donor; SA: splicing acceptor).

FIG. 2 shows a graph in which vitality of LAK cells is compared with that of I-LAK cells of the present invention.

FIG. 3 shows a graph in which cytotoxicity of LAK cells on a variety of target cells is compared with that of I-LAK cells of the present invention.

FIG. 4 shows a graph in which HIV production is quantified by EIA assay following co-cultivation of LAK cells or I-LAK cells and HIV-infected cells.

### SUMMARY OF THE INVENTION

The present invention provides a transformed lymphokine activated killer cell into which a recombinant vector carrying IL-2 gene to which it is operably linked is introduced. This transformed cell is hereinafter referred to as I-LAK cell.

Preferably, I-LAK cell of the present invention is one transfected with a retroviral vector carrying IL-2 to which it is operably linked.

More preferably, I-LAK cell of the present invention is one transfected with a retroviral vector LNC/IL-2/IRES/TK.

In another aspect, the present invention provides a process for producing I-LAK cell useful in the treatment of cancer or virus infection which comprises (a) collecting peripheral blood from mammalian animals and removing erythrocytes from peripheral blood to obtain lymphocytes-containing leukocytes fraction, (b) culturing the resulting lymphocyte-containing leukocyte fraction in a IL-2-containing medium to generate LAK cells, (c) transfecting the resulting LAK cell with a recombinant vector carrying IL-2-secreting gene to which it is operably linked and culturing the transfected cells in a medium and under conditions appropriate to differentiate them and (d) screening and recovering cells with ability to secrete IL-2 from culture solution.

In another aspect, the present invention provides a pharmaceutical composition for treating cancer or virus infection disease comprising a sufficient amount of I-LAK cells to elite immune response.

### DETAILED DESCRIPTION OF THE INVENTION

One embodiment of the present invention includes I-LAK cell into which a recombinant vector carrying IL-2 gene to which it is operably linked is introduced. I-LAK cell of the present invention is characterized in that it constantly maintains low level of IL-2 protein in vivo by continuously secreting a regular amount of IL-2 and thereby selectively destroys cancer cells or virus-infected cells while does not affect normal cells. Once I-LAK cells of the present invention are administered into mammalian animals suffering from cancer or virus infection, they continuously secrete low concentration of IL-2 protein in vivo and as results the low concentration of secreted IL-2 protein retains the cytotoxicity of I-LAK cells on cancer cells or virus-infected cells while hardly affecting normal cells.

LAK cells are non-specific immune effector cells capable of digesting virus-infected cells and various transformed cells through non-MHC-restriction mechanism. LAK cells are produced by culturing peripheral blood mononuclear cells with IL-2 proteins and can be used in the treatment of cancer, virus-infection disease, autoimmune disease, etc.

U.S. Pat. No. 4,690,915 describes that the combined administration of LAK cell and IL-2 is effective in the treatment of malignant melanoma, lung cancer, renal cancer, colon cancer and rectal cancer. Also, it was described that LAK cells have various tumor-killing activity against colon cancer, pancreas cancer, esophageal cancer and etc. (Rayner et al., Cancer, 55, 1327-1333(1985)).

IL-2 protein is a potent immunoregulatory cytokine as lymphokine capable of promoting the in vitro long-term culture of antigen specific effector T-lymphocyte (Morgan et al., Science, 193, 1007-1008 (1976)). IL-2 protein is produced by CD4 T-lymphocyte and very few quantity of IL-2 by CD8 T-lymphocyte. IL-2 protein functions as an autocrine growth factor that stimulates the secretory cell itself. Also, IL-2 protein functions as a paracrine growth factor as it affects adjacent T-lymphocyte including CD4 and CD8 cells. It is also known that IL-2 protein regulates a variety of immunological functions including the influence on cytotoxic T cells, NK cells and activated B cells and enhances the cytolytic function by generating LAK cells.

IL-2 gene introduced into LAK cells according to the present invention is responsible for innate activity of IL-2 protein to enable LAK cells to keep alive in the body. cDNA encoding human IL-2 protein has been cloned (Taniguchi et al., Nature, 302, 305 (1983)) and the amino acid sequence was deduced from it. In eukaryotic cells, IL-2 protein is first produced as a precursor polypeptide consisting of 153 amino acid residues and then, following the deletion of 20 amino acid residues, is modified into a mature IL-2. Human recombinant IL-2 with innate activity has been produced in E. coli (Rosenberg et al., Science, 223, 1412 (1984)), insect cells (Smith et al., Proc. Natl. Acad. Sci. U.S.A., 82, 8404 (1985)) and mammalian COS cell (Taniguchi et al., Nature, 302, 305 (1983)) using molecular biology techniques.

IL-2 gene introduced into LAK cells according to the present invention can be originated from any suitable sources including known recombinant IL-2 genes. Specifically, IL-2 gene includes natural and recombinant IL-2 genes and biologically functional equivalents thereof. The biologically functional equivalents mean polypeptides having the same or very similar biological activity, such as the rIL-2 muteins described in U.S. Pat. No. 4,518,584 and rIL-2 proteins having a methionine replacing the NH₂-terminal alanine.

As used herein, the term “low concentration of IL-2 protein” or “low level of IL-2 protein” means an amount of IL-2 protein which is capable of stimulating and activating LAK cells without adverse side effects such as the temporarily increased induction of virus activity and cytokine leak syndrome. Preferred amounts of IL-2 protein daily secreted from LAK cells in a human body are about 500,000 IU or below. As mentioned above, it has been suggested that immunotherapy using LAK cells needs the injection of high concentration of IL-2 protein in combination with LAK cells in order to keep survival of LAK cells. However, the systematic administration of high concentration of IL-2 protein leads to increase of LAK cells having the secondary latent HIV virus or results in leakage of intravascular fluid into extravascular space (capillary vessel leak syndrome) (Rosenstein M. et al., Immunology, 137, 1735-1742 (1986); Ohkubo C. et al., Cancer Res., 51, 1561-1563 (1991); Edwards, M. J. et al., Cancer Res., 52, 3425-3431 (1992); Damle N. K. et al., J. Immunol., 142, 2660-2669 (1989)). The capillary vessel leak syndrome caused by administration of IL-2 protein includes, for example, formation of edema, hypotension and renal dysfunction. Where IL-2 is administered to humans for the treatment of tumor, no more than 10% of optimal dose obtained through animal test are tolerable. However, even routine dose can result in inertia, gastric lavage, emesis, diarrhea, hypotension and organ dysfunction, not so much as death.

As used herein, the term “cancer cell” means that it divides and reproduces abnormally with uncontrolled growth, unlike normal cells, and can break away surrounding tissue and grow into infiltration. Examples of cancer cell include pancreatic cancer cell, lung cancer cell, colon cancer cell, liver cancer cell, breast cancer cell, prostate cancer cell, bladder cancer cell, skin cancer cell and soft tissue cancer cell. Preferred cancer cells include malignant melanoma cell, lung cancer cell, kidney cancer cell, colon cancer cell and rectal cancer cell.

As used herein, the term “irus-infected cell” means that normal cell is mutationally altered by infection with virus and has the following properties. It is easily grown ex vivo. Its growth is faster than that of normal cell. A lot of changes occur on cellular surface, for example, increase of the ability of ions to pass, loss of toxic hormone to bind, generation of new antigen, etc. The chromosomes are altered and antiviral agents such as interferon are formed. Especially, infection by virus herein means that normal cells are infected with human immunodeficiency virus (HIV). HIV is a casual agent of acquired immunodeficiency syndrome, called AIDS. HIV's main target for an attack is helper T cell among T cells regulating immune function. Once helper T cell is infected with HIV and develops into necrosis, human immune function is damaged and immunodeficiency occurs, resulting in fatal infection and malignant tumor.

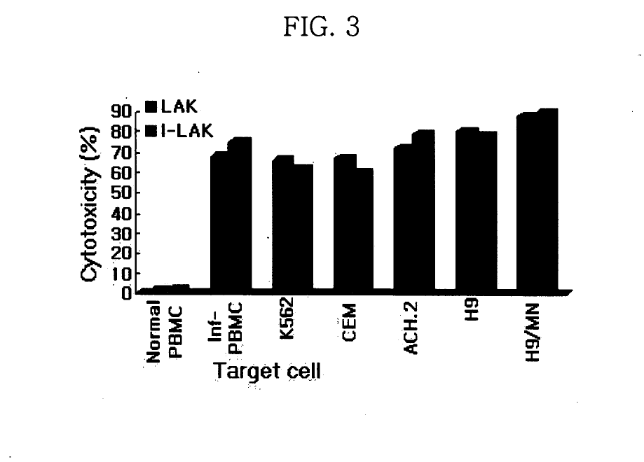

As used herein, the term “selectively destroy” or “selectively kill” means that I-LAK cell of the present invention exhibits cytotoxicity against cancer cells or virus-infected cells but not normal cells and as results lyses and removes said cells. We investigated cytotoxicity of I-LAK cells on a variety of cells including HIV-infected peripheral blood mononuclear cell, K562, CEM, ACH.2, H9, H9/HTLV-III_{MN}, etc. and found that I-LAK cells selectively kill HIV-infected cells and malignant cells (Example 5, FIG. 3) but do not affect normal cells.

I-LAK cell of the present invention has the following properties:

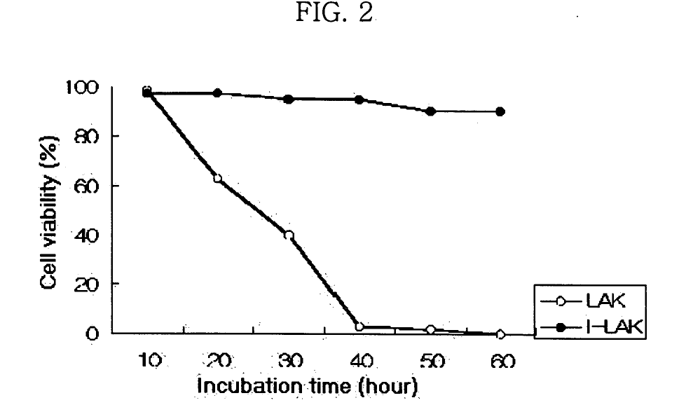

1) LAK cells are dependent on IL-2 protein, i.e., can be survived in the presence of IL-2 protein (Grimm E A et al., J Exp Med 158(4), 1356-61 (1983)). As contrast, I-LAK cells carrying IL-2 gene can keep alive without IL-2 protein being added. This fact was first confirmed by the inventors and is based on the finding that when LAK cells and I-LAK cells were separately cultured in the absence of IL-2 protein for 60 days, the viability of LAK cells and I-LAK cells are 0% and about 90% at day 60 of culture, respectively (Example 4, FIG. 2).

2) IL-2 protein secreted from I-LAK cells stimulates HIV-infected cell and help it escape on the latent period, reducing a total number of latent cells in human body (Chun T W et al., J. Exp. Med. 188(1), 83-91 (1998. 12)).

3) I-LAK cells secrete IL-2 protein in low concentration, preventing instant activation of virus. It has been reported that such instant activation of virus would occur when LAK cells and high concentration of IL-2 protein were administered to HIV-infected person according to the prior art (Ramratnam B. et al., Nat. Med. 6(1), 82-5 (2000. 09)).

4) I-LAK cells constantly secrete IL-2 protein, enhancing the immunity of HIV-infected person. It has been suggested that IL-2 protein chronically increases the number of cells critical to the immune of CD⁴⁺ T cells (Davey R T Jr. et al., JAMA, 284(2), 92074 (2000)).

Another embodiment of the present invention provides a process for producing I-LAK cell into which a recombinant vector carrying IL-2 gene to which it is operably linked is introduced which comprises (a) collecting peripheral blood from mammalian animals and removing erythrocytes from peripheral blood to obtain lymphocytes-containing leukocytes fraction, (b) culturing the resulting lymphocyte-containing leukocyte fraction in a IL-2-containing medium to generate LAK cells, (c) transfecting the resulting LAK cell with a recombinant vector carrying IL-2-secreting gene to which it is operably linked and culturing the transfected cells in a medium and under conditions appropriate to differentiate them and (d) screening and recovering cells with ability to secrete IL-2 from culture solution.

More particularly, peripheral blood is collected from mammals and centrifuged. Ficoll-Paque is added and again centrifuged to afford peripheral blood mononuclear cells. The resulting peripheral blood mononuclear cells are suspended in LAK-activating medium containing IL-2 protein to produce LAK cells.

The LAK cells obtained thereby are transfected with retrovirus vector, preferably recombinant retrovirus vector LNC/IL-2/IRES/TK to simultaneously express multiple genes, i.e., IL-2 gene and thymidine kinase gene of herpes simplex virus. The transfected cells are cultured in a medium containing G418 (neomycin analog) to obtain G418-resistant cells which are cultured again. The supernatant of the culture is subject to the detection of IL-2 protein, for example ELISA, to screen cells with the ability to highly secrete IL-2 protein. The screened cell was designated as I-LAK cell.

The present invention exemplifies plasmid LNC/IL-2/IRES/TK as a recombinant vector carrying IL-2 gene. This recombinant vector can be readily made as known and also be obtained without restriction from Dr. Kim, Yeon-Soo of Korea Advanced Institute of Science and Technology, Daejeon, Korea.

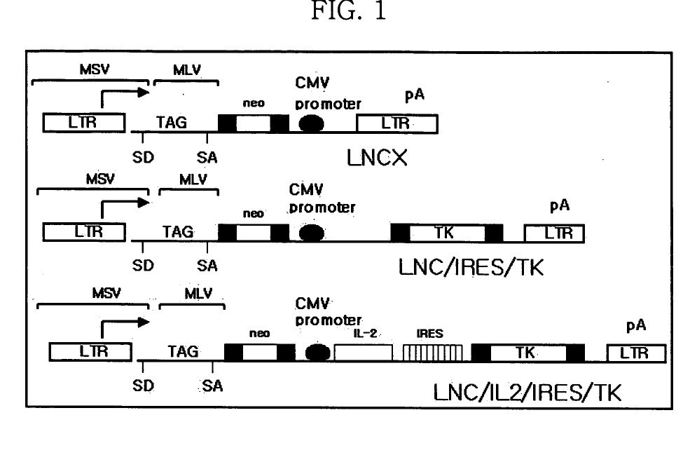

The recombinant vector LNC/IL-2/IRES/TK carries IL-2 gene under CMV promoter and thymidine kinase (TK) gene. TK gene makes it possible to treat transfected cells with recombinant vector with antiviral agents. The expression product of TK gene remarkably enhances the sensitivity of cells to ganciclovir. Preferably, LTR promoter/neomycin marker/CMV promoter, IL-2 gene, internal ribosome entry site gene and tyrosine kinase gene are operably linked in order (FIG. 1).

As used herein, the term “recombinant vector” refers to a nucleic acid molecule capable of directing the expression of another nucleic acid molecule, i.e., IL-2 gene, to which it is operably linked. The recombinant vector of the invention includes IL-2 gene in a form suitable for expression of the nucleic acid in a host cell, which means that the recombinant vector include one or more regulatory sequences, selected on the basis of the host cells to be used for expression, which is operatively linked to the nucleic acid sequence coding for IL-2 to be expressed. One type of recombinant vector is capable of autonomous replication in a host cell which it is introduced. Another type of recombinant vector is integrated into the genome of a host cell upon introduction into the host cell, and thereby is replicated along with the host genome. Mammalian cells are used as a host cell in the present invention.

A variety of vector, which generally refers to a nucleic acid molecule capable of transporting another nucleic acid to which it has been linked, can be used in the present invention. Examples of the virus vector suitable for the present invention include SV40, bovine papilloma virus, retrovirus, adenovirus, herpes simplex virus, poxvirus, lentivirus, adeno-associated virus and cytomegalovirus.

Retrovirus vector is especially preferred in the present invention. The retrovirus is any virus in the family Retroviridae that has RNA as its nucleic acid and uses the enzyme reverse transcriptase to copy its genome into the DNA of the host cell chromosomes. The retrovirus vector derived from MoMLV (Moloney Murine Ieukemia Virus) is most available in the art. The retrovirus vector is advantageous in that the in vitro handling is convenient, the delivery of genes is efficient, and the integration into the host cell chromosomes is stable so that it can be constantly transferred to progeny cells. The LNCX retrovirus vector exemplified in the invention is characterized in that neo^{r} marker gene is expressed under 5′ LTR promoter and immediate-early promoter of human cytomegalovirus (CMV) is included as internal promoter, resulting in high expression of the foreign gene.

As used herein, the term “regulatory sequence” means DNA sequence essential for the expression of the coding genes to which it is operably linked in certain host cells. It is intended to include promoters to which RNA polymerase binds and initiates transcription, optionally operators capable of controlling such transcription, sequence coding for mRNA ribosome binding site and sequences regulating termination of transcription and translation. Examples of regulatory sequence for eukaryotic cells include promoter, polyadenylation signal, enhancer, etc.

Within a recombinant vector, the term “operably linked” is intended to mean that the nucleotide sequence of interest is linked to the regulatory sequence(s) in a manner which allows for expression of the nucleotide sequence when appropriate molecules (for example, transcription activating protein) are bound to the regulatory sequence. For example, DNA to pre-sequence or leader is operably linked to DNA to polypeptide in a case where it is expressed as preprotein that participates in secretion of polypeptide; promoter or enhancer is operably linked to coding sequence in a case where it influences on transcription; or ribosome binding site is operably linked to coding sequence in a case where it influences on transcription; or ribosome binding site is operably linked to coding sequence in a case where it is positioned so as to facilitate translation. Generally, “operably linked” means that DNA sequences are contiguous and leader sequence is within a reading frame. However, enhancer is not required to be contiguous. The linking of these sequences is performed by ligation at convenient restriction sites. However, if such restriction sites do not exist, synthetic oligonucleotide adaptor or linker is used according to conventional methods.

The recombinant vector can be introduced into a host cell by conventional transformation methods such as DEAE-dextran, potassium phosphate and electroporation. The techniques for transforming host cells and expressing cloned foreign DNA sequence in the transformed cells are well known to those skilled in the art (Maniatis et al., Molecular Cloning: A Laboratory Manual, Cold Spring Harbor Laboratory (1982); Sambrook et al., Molecular Cloning: A Laboratory Manual, Cold Spring Harbor Laboratory (1989); Gene Expression Technology, Method in Enzymology, Genetics and Molecular Biology, Methods in Enzymology, Guthrie & Fink (eds.), Academic Press, San Diego, Calif. (1991); Hitzeman et al., J. Biol. Chem., 255, 12073-12080 (1980); and U.S. Pat. No. 4,935,349). Preferably, the recombinant vector of the present invention is transformed into a host cell by potassium phosphate precipitation.

Another embodiment of the present invention provides a pharmaceutical composition for treating cancer or virus infection disease comprising a sufficient amount of I-LAK cells carrying IL-2 gene to elicit immune response.

A pharmaceutical composition of the present invention can include pharmaceutically acceptable carriers. Especially, various carriers or diluents including brine, buffered brine and mixture of brine with nonspecific serum albumin can be used in the present invention. The pharmaceutical composition can contain, for example, additives, buffers, antioxidants, carbohydrates such as glucose, sucrose and dextrine, and chelating agents such as EDTA. Additionally, the pharmaceutical composition can include water, saline, glycerol, ethanol, emulsifying agents, wetting agents or pH-adjusting agents. The pharmaceutical composition can also include adjuvant for enhancement of immune response. Examples of adjuvant include, but are not limited to, aluminum hydroxide (alum), thr-MDP, nor-MDP and MPT-PE.

A pharmaceutical composition of the present invention can be administered parenterally, intramuscularly, subcutaneously, intradermally, intraperitoneally, intranasally or intravenously or via other routes suitable for the treatment of tumor or infection and the conditions of patients. Since low level of inflammation or convulsion can occur, it is preferred to choose relatively non-invasive methods. Especially preferred is a subcutaneous route.

A pharmaceutical composition of the present invention is administered at the sufficient amount to elicit immune response to the subject. The dose of the pharmaceutical composition of the present invention is typically about 10⁵ to 10¹¹ I-LAK cells, preferably about 10⁶ to 10¹⁰ I-LAK cells and more preferably about 1×10⁷ to 2×10⁹ I-LAK cells. However, the dose can be varied depending on the degree to which protection is required, age or weight of the subject, formulation type of the composition, administration method, etc.

The present invention will now be explained on the basis of some specific embodying examples, which, however, are not to be considered as limiting.

### EXAMPLES

### Example 1

Preparation of LAK Cells

Peripheral blood was collected from HIV-negative healthy donor (HIV antibody negative) by venipuncture. Ficoll-Paque was added to the peripheral blood and the resulting mixture was centrifuged to obtain peripheral blood mononuclear cells. The peripheral blood mononuclear cells were cultured in RPMI-1640 medium supplemented with 15% fetal bovine serum (FBS), penicillin, streptomycin and L-glutamine. The resulting culture solution was treated with 1,000 IU/ml of rIL-2 (aldesleukin, Chiron BV Amsterdam, Netherland) to produce LAK cells.

### Example 2

Construction of Recombinant Vector LNC/IL-2/IRES/TK

In order to introduce IL-2 gene into LAK cells obtained by Example 1, a multiple gene expression retroviral vector LNC/IL-2/IRES/TK was constructed so as to simultaneously express IL-2 gene and thymidine kinase gene (HSV TK gene) of herpes simplex virus.

The recombinant vector LNC/IL-2/IRES/TK was constructed by sequentially inserting HSV TK gene, internal ribosome entry site (IRES) and IL-2 gene into retrovirus vector LNCX as shown in FIG. 1.

The fragment of HSV TK gene with the deletion of promoter and poly(A) signal sites was synthesized by PCR from pTK3 (BRL, Gaithersburg, Md.). Both ends of the synthetic fragment were made as phosphorylated blunt ends. The resulting blunt-ended fragment was inserted into retrovirus vector LNCK which had been digested with restriction endonuclease HpaI. LNCX retrovirus vector includes neo^{r} marker gene, which is expressed under 5′ LTR promoter, and immediate-early promoter of human cytomegalovirus (CMV promoter) as an internal promoter and therefore is expected to highly express the foreign genes. The resulting recombinant vectors were analyzed by restriction digestion and nucleotide sequencing to screen LNC/TK clones in which HSV TK gene is expressed under CMV promoter. About 600 bp EcoRI-NcoI fragment was excised from plasmid pCITE-1 (Novagen, Wis.) and subcloned into the recombinant vector LNC/TK so that cap-independent translation enhancer sequence including internal ribosome entry site (IRES) of encephalomyocarditis virus (EMC) is introduced upstream of HSV-TK gene. The resulting subclone is designated as LNC/IRES/TK. About 700 bp HindIII-BamH1 IL-2 DNA fragment (Invivogen, USA) was subcloned between CMV promoter and IRES sequence to construct retroviral vector LNC/IL-2/IRES/TK.

### Example 3

Preparation of Retrovirus Packaging Cell Line

Plasmids LNC/IRES/TK or LNC/IL-2/IRES/TK were transfected into amphotropic packaging cell line PA317 (ATCC, USA) by calcium phosphate coprecipitation to produce retroviral packaging cell line producing retrovirus encoding HSV TK gene and/or IL-2 gene. After 48 hours, the medium was changed to a fresh medium containing 600 ug/ml of G418 and it was cultured during 10 to 14 days to form G418-resistant colonies. The resulting colonies were isolated and enriched by mass culture. Cultures of PA317/LNC/IRES/TK cell and PA317/LNC/IL-2/IRES/TK cell were separately diluted in order. 5×10⁵ NIH/3T3 cells were cultured on Petri dishes having a diameter of 60 mm for 24 hours. 1 ml of the culture was inoculated on each dilution obtained above which was infected for 4 hours. 4 ml of a fresh culture solution was added and it was cultured for 48 hours. The cultured cells were treated with trypsin and inoculated on Petri dishes having a diameter of 100 mm which were cultured in culture solution containing 400 ug/ml of G418 for 10 to 14 days. The above experiment was repeated three times. The virus titer was determined from the results of the three experiments and cell lines with highest virus titer were selected.

### Example 4

Preparation of I-LAK Cells

On 10 day of culture following production of LAK cells, LAK cells were transformed with retroviral vector LNCX/IL-2/IRES/TK having CMV promoter to produce I-LAK cells. The retroviral vector was added to LAK cells. After 48 hours, the cells were washed and cultured on RPMI-1640 containing G418 (neomycin analog) so that cells carrying neo^{r} gene could be grown. G418-resistant cells were obtained and cultured. The culture supernatant was measured by ELISA to detect the ability of the cells to secrete IL-2. I-LAK cells were confirmed on the inclusion of IL-2 gene by RT-PCR.

### Example 5

Determination of Viability of LAK Cells and I-LAK Cells

LAK cells and I-LAK cells were cultured on a medium without cell stimulants for 60 days. The viability of cells was measured by trypan blue exclusion. As the experiment results, the vitality of LAK cells was 98% on day 10 of culture, 63% on day 20 of culture, 40% on day 30 of culture, 3% on day 40 of culture, 2% on day 50 of culture and 0% on day 60 of culture. As contrast, the vitality of I-LAK cells was 97% on day 10 of culture, 97% on day 20 of culture, 95% on day 30 of culture, 95% on day 40 of culture, 90% on day 50 of culture and 90% on day 60 of culture (FIG. 2).

### Example 6

Determination of Cytotoxicity of LAK Cells and I-LAK Cells on Various Target Cells.

The cytotoxicity of LAK cells and I-LAK cells on a variety of target cells was determined by ⁵¹chromium release assay.

Normal peripheral blood mononuclear cells, HTLV-III_{MN}-infected peripheral blood mononuclear cells, KS62, CEM, ACH.2, H9 and H9/HTLV-III_{MN} were used as target cells. KS62, target cell line of LAK cell, was used as positive control in LAK cytotoxicity assay. CEM cell line was used to compare the level of LAK cell activity of CD4+ human cell line ACH.2. ACH.2 is HIV-1-latently infected T cell subclone A3.01 which was derived from HIV_{LAV}-CEM. The above human T cell line was isolated from 4 year-old caucasin female patient suffering from acute lymphoblastic lymphoma. H9 and H9/HTLV-III_{MN} are mononuclear clones derived from specific HUT 78 cell line. ACH.2, H9 and H9/HTLV-III_{MN} were obtained from NIH AIDS Research and Reference Regent Program. All of the above cells were cultured in RPMI-1640 supplemented with 1% FBS, penicillin, streptomycin and L-glutamine.

The above target cells (3×10⁶) were cultured in 0.5 ml of medium containing 150 ul of ⁵¹Cr in a water bath at 37° C. ⁵¹Cr-labeled cells were washed four times with fresh media and 50 ul of cells were streaked on 96-well plate at 10⁴ cells/well. LAK cells and I-LAK cells were added to the plate at the ratios of 20:1, 1:1 and 1:20. The effector and target cells were cultured in 5% CO₂ cultivator at 37° C. for 8 hours. 50 ul of supernatant was taken from each well and counted in isotope counter. These experiments were repeated three times and percentage of lysis was calculated by the following formula:

$$Percentage of lysis ( \% ) = \frac{Experimentanl release - spontaneous release }{Maximal release - spontaneous release } \times 100$$

The maximal release was determined by culturing 50 ul of 0.5% trypton X-100 and 50 ul of target cells. The spontaneous release was determined by culturing 50 ul of cell culture solution and 50 ul of target cells. The spontaneous release was always not more than 20% of the maximal release (Antonelli P et al., Clin. Immunol. Immunopath. 19, 161-169 (1981)). A variety of parameters between LAK cells and I-LAK cells was compared by using student's T-test. When p was 0.05 or below, it was considered statistically significant.

The results reveal that LAK and I-LAK cells were potently cytotoxic to all tumor cells, K562, CEM, ACH.2, H9 and H9/HTLV-III_{MN}. This is considered as being that LAK cells are implicated in the destroy of malignant cells and virus-infected cells. All of the malignant cells and HIV-infected cells were selectively killed and removed, whereas normal cells were not damaged. Although relatively statistic difference was not found, the specific lyses of ACH.2 cells and H9/HTLV-III_{MN} were higher than those of CEM cells and H9 cells, respectively. Meanwhile, there was no significant difference between selective cytotoxicity of LAK and I-LAK cells on HIV-infected cells (FIG. 3).

### Example 7

Comparison of HIV Production in Co-Culture of LAK Cells or I-LAK Cells with HIV-Infected Cells

In order to prepare HIV-infected peripheral blood mononuclear cells, HIV virus stock was produced and then peripheral blood cells were infected with the HIV virus stock. Infected H9 cells were cultured at the time when they produced highest virus titers. The supernatant was recovered from the resulting culture solution to afford the HIV virus (HTLV-III_{MN}) stock. The titer of the HIV virus stock was measured by determining TCID₅₀ endpoint 7 days after cells were infected with the HIV virus stock. HIV infection of peripheral blood cells was induced by culturing with a moi (multiplicity of infection) of 1.0 at 37° C. for 12 hours. Control cells were obtained by culturing the supernatant of non-infected H9 cell culture. Infected peripheral blood cells were centrifuged, washed and suspended in a fresh medium. A half of the culture supernatant was replaced with a fresh medium at interval of 3 days. The viability of cells was measured by trypan blue exclusion.

LAK cells or I-LAK cells were co-cultured with HIV-infected peripheral mononuclear cells obtained above and the amount of virus in cell culture solution was quantified at interval of 3 days using HIV-1 p24 antigen-capture EIA kit (Organon Teknika, Boxtel, Netherlands). A standard curve was prepared per experiment and the amount of p24 was calculated on the basis of number of specimen dilutions, standard curves and OD values.

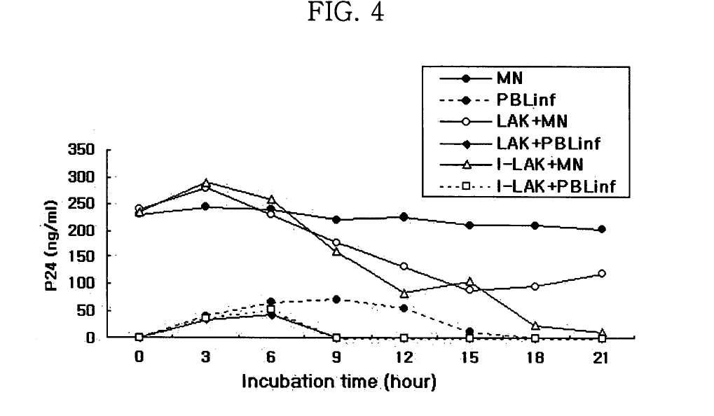

It could be seen from the quantification of HIV p24 antigen isolated from the culture supernatant of H9/HTLV-III_{MN} cell line (MN) that the most virus replication happened on day 6 of culture. In a case where H9/HTLV-III_{MN} cell line was co-cultured with LAK cells, the value of p24 antigen was declined until day 15 of culture and rose from day 18 of culture. It is considered that the rise of p24 antigen value (rally of virus replication) is due to the reduction of the viability of LAK cells at the time of from the second week to the third week for which IL-2 stimulant was depleted. For co-culture of H9/HTLV-III_{MN} cell line and I-LAK cell, the value of p24 antigen was declined steadily until about day 21. The co-culture of peripheral blood cell infected with H9/HTLV-III_{MN} cell line resulted in the rise of virus replication until day 6 of culture. The co-culture of HIV-infected peripheral blood cell (PBLinf) and LAK cell or I-LAK cell resulted in the notable decline of p24 antigen value between days 9 to 21 of culture. The in vitro results show that the ability of LAK cell to kill HIV-infected cell is almost equal to that of I-LAK cell (FIG. 4).

### Industrial Applicability

As I-LAK cell of the present invention exhibits activity to selectively kill cancer cell or virus-infected cell while does not affect normal cell by secreting low concentration of IL-2 protein, it is useful in the treatment of cancer and virus infection.

From the foregoing detailed description it will be evident that modifications can be made by those skilled in the art without departing from the spirit or scope of the invention. In the connection with this, it should be understood that the foregoing examples are for illustrative purposes but not for limit of the present invention. Therefore, it is intended that all modifications and variations not departing from the spirit of the invention come within the scope of the claims and their equivalent.

1. A transformed lymphokine activated killer cell (I-LAK cell) useful in the treatment of cancer or virus infection into which a recombinant vector carrying IL-2 gene to which it is operably linked is introduced.

2. The I-LAK cell of claim 1 wherein said recombinant vector comprises retrovirus vector.

3. The I-LAK cell of claim 2 wherein said retrovirus vector is vector LNC/IL-2/IRES/TK.

4. A process for producing a transformed lymphokine activated killer cell (I-LAK cell) useful in the treatment of cancer or virus infection into which a recombinant vector carrying IL-2 gene to which it is operably linked is introduced which comprises (a) collecting peripheral blood from mammalian animals and removing erythrocytes from peripheral blood to obtain lymphocytes-containing leukocytes fraction, (b) culturing the resulting lymphocyte-containing leukocyte fraction in a IL-2-containing medium to generate LAK cells, (c) transfecting the resulting LAK cell with a recombinant vector carrying IL-2 gene to which it is operably linked and culturing the transfected cells in a medium and under conditions appropriate to differentiate them and (d) screening and recovering cells with ability to secrete IL-2 from culture medium.

5. A pharmaceutical composition for treating cancer or virus infection disease comprising a sufficient amount of I-LAK cell of claim 1 to elicit an immune response.

6. The pharmaceutical composition of claim 5 wherein said cancer comprises malignant melanoma, lung cancer, renal cancer, colon cancer and rectal cancer.

7. The pharmaceutical composition of claim 5 wherein said virus comprises HIV virus.
